(19) United States
(12) Patent Application Publication
(10) Pub. No.: US 2026/0009166 A1
(43) Pub. Date: Jan. 8, 2026

(54) APPARATUS AND PROCESS FOR RECYCLING COTTON AND COTTON/POLYESTER CONTAINING TEXTILES

(71) Applicant: 14787285 Canada Inc. dba Nuria Scientific, St Catharines (CA)

(72) Inventors: Nicholas Leroy Ledra, Thorold (CA); Wankei Wan, St Catharines (CA)

(21) Appl. No.: 18/761,588

(22) Filed: Jul. 2, 2024

(51) Int. Cl. D01G 11/00; C08J 11/10; C12N 9/24; D02G 3/02

(52) U.S. Cl. CPC D01G 11/00; C08J 11/105; C12N 9/2402; D02G 3/02; C08J 2301/02; C08J 2367/02; D10B 2201/02

(57) ABSTRACT

A process for recycling of used textile. The process includes the incorporation of textile into a bioreactor containing at least one enzyme with a solvent for incubation, wherein: the textile includes at least cotton. The at least one enzyme biochemically reacts with cellulose in the cotton and converts the cotton into cotton yarn for reuse in textile manufacturing. The process feed may include cotton containing blends, such as those containing cotton and polyester, whereby the process includes an additional step of separating the polyester from the cotton by dissolution for recovery. The recovered polyester is reprocessed for textile use.

### FIELD OF THE DISCLOSURE

This disclosure relates to recycling of textiles in general, and more particularly, to recycling of cotton and cotton/polyester (PET) containing textiles.

### BACKGROUND

Textiles (e.g., fibers, threads, yarn, fabrics, fragments of textile, etc.) are materials made of natural or synthetic fibers, such as cotton and PET. Examples of products fabricated from textiles include clothing, carpets, bedding, purses, tablecloths, drapery, window shades, towels, geotextiles, etc. At end-of-life of a textile, the textile can either be disposed or recycled. Current processes of disposing textiles includes disposal of textiles at landfills and the incineration of textiles. However, disposal of textiles in landfills, such as synthetic fibers, can take hundreds of years to decompose, releasing greenhouse gases and leaching toxic substances into the soil and groundwater. While incineration reduces waste volume and generates energy, it can produce toxic emissions and is less favored due to environmental concerns. Recycling of textiles provides a more sustainable and environmentally friendly alternative to disposing of textiles. Current processes for recycling textiles encompass a variety of approaches, such as mechanical recycling, thermal recycling, and chemical recycling (see, U.S. Pat. Nos. 10,569,900B2, 10,843,897B2, US20140322719A1, US20190225264A1, US20200165747A1, WO2021028485A1).

Mechanical recycling involves the physical breakdown of textiles. This process includes shredding fabrics into fibers. While this process is effective at breaking down textiles, mechanical recycling often degrades the quality of fibers, making them shorter and weaker. This limits the usability of recycled fibers, typically restricting them to low-grade applications like insulation or stuffing. Further, effective mechanical recycling can be labor-intensive and costly when separation of different types of textiles, particularly those blended with multiple fibers, is required. In cases where textiles contain dyes, finishes, and other contaminants, mechanical recycling becomes more complicated and the quality of the final product is reduced.

Thermal recycling involves the use of heat to convert textiles into energy or new materials. Pyrolysis, for example, breaks down organic materials in the absence of oxygen to produce synthetic gas, oil, and char. However, thermal recycling processes, like pyrolysis, require substantial amounts of energy, which can be a significant environmental and economic cost. Burning textiles, especially synthetic ones, can also release harmful pollutants and greenhouse gases, contributing to air pollution and climate change. Further, thermal processes typically result in the production of basic chemicals or energy rather than reusable fibers, failing to contribute to the circular economy for textiles.

Chemical recycling breaks down textiles at a molecular level to recover fibers. This process is advantageous as it can handle a wider variety of textiles, including mixed-fiber fabrics, and often produces higher-quality recycled fibers. However, known chemical recycling processes are more costly and complex compared to mechanical recycling due to the need for specialized facilities, chemicals, and processes. In fact, because of its complexity, the technology required for chemical recycling is still under development for many types of textiles, limiting its widespread adoption. Further, known chemical recycling processes often involve the use of harsh chemicals and significant energy consumption, which can offset some environmental benefits.

In some instances, everyday products are processed to recover fibers for textile manufacturing. For instance, the prior art teaches the processing of water bottles to recover PET fibres (see, GAYASKIN (2020). “Polyester recyclé: matière écologique, mais pas à n′importe quel prix.”). Recovering PET fibres from water bottles involves collecting, sorting, cleaning, and shredding used bottles into flakes. These flakes are then melted and extruded into pellets or fibers, which can be used to produce new bottles and polyester textiles. This process reduces plastic waste, conserves natural resources, and lowers energy consumption and greenhouse gas emissions compared to producing virgin PET.

While current processes for recycling textiles and recovering fibers for use in textile manufacturing offer some solutions, they each come with significant drawbacks that need to be addressed to create a more sustainable textile industry. An improved cost-effective, efficient, and environmentally friendly process for recycling textiles is proposed.

### SUMMARY

The following presents a simplified summary of some embodiments of the techniques described herein in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented below.

Some aspects include a process for recycling a textile, including: combining at least one enzyme with an aqueous solvent, their combination resulting in a creation of a solution; depositing the solution within a bioreactor; suspending the textile in the solution within the bioreactor for an incubation period, wherein: the textile includes at least cotton; during the incubation period the at least one enzyme biochemically reacts with cellulose of the cotton in the textile, thereby breaking down the cellulose and producing other products; and cotton yarn, the solution, and the other products are output from the bioreactor after the incubation period; filtering the cotton yarn, the solution, and the other products output from the bioreactor after the incubation period through a first filter, separating at least the other products from the cotton yarn and at least some of the solution; recovering at least a first portion of the cotton yarn filtered through the first filter; and returning any portion of the cotton yarn and the at least some of the solution filtered through the first filter back to the bioreactor when further processing of the any portion of the cotton yarn is required.

Some aspects provide an apparatus for recycling a textile, including: a bioreactor configured for incubation of the textile and a solution including at least one enzyme and an aqueous solvent within which the textile is suspended for an incubation period at a temperature and a pressure, wherein: the textile includes at least cotton; during the incubation period the at least one enzyme biochemically reacts with cellulose of the cotton in the textile, thereby breaking down the cellulose and producing other products; and cotton yarn, the solution, and the other products are output from the bioreactor after the incubation period; a first filter configured to filter the cotton yarn, the solution, and the other products output from the bioreactor after the incubation period, separating at least the other products from the cotton yarn and at least some of the solution; wherein: at least a first portion of the cotton yarn filtered through the first filter are recovered; and any portion of the cotton yarn and the at least some of the solution filtered through the first filter are returned to the bioreactor when further processing of the any portion of the cotton yarn is required.

### BRIEF DESCRIPTION OF THE FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates an example of a process for recycling a cotton containing textile, according to some embodiments.

FIG. 2 illustrates an example of a process for recycling a cotton/PET containing textile, according to some embodiments.

FIG. 3 illustrates an example of an apparatus for recycling the cotton containing textile as described in FIG. 1, according to some embodiments.

FIG. 4 illustrates an example of an apparatus for recycling the cotton/PET containing textile as described in FIG. 2, according to some embodiments.

### DETAILED DESCRIPTION OF SOME EMBODIMENTS

The present inventions will now be described in detail with reference to a few embodiments thereof as illustrated in the accompanying drawings. In the following description, numerous specific details are set forth in order to provide a thorough understanding of the present inventions. It will be apparent, however, to one skilled in the art, that the present invention may be practiced without some or all of these specific details. In other instances, well known process steps and/or structures have not been described in detail in order to not unnecessarily obscure the present invention. Further, it should be emphasized that several inventive techniques are described, and embodiments are not limited to systems implanting all of those techniques, as various cost and engineering trade-offs may warrant systems that only afford a subset of the benefits described herein or that will be apparent to one of ordinary skill in the art.

Some embodiments provide an efficient and environmentally friendly apparatus and process for recycling a cellulose-derived textile, in particular, a cotton and a cotton/PET containing textile. Cellulose is primarily derived from plants, such as wood and cotton. Cellulose may also be extracted from other sources such as algae, bacteria, and fungi. Some cellulose is too impure for direct use in textiles (e.g., wood derived cellulose) and may be used for other applications. For instance, wood pulp from wood is processed to extract cellulose that may be used for producing paper. In some cases, cellulose unusable for textile manufacturing may undergo a process to produce regenerated cellulose usable for textiles. Unlike cellulose derived from wood, bacteria, and other cellulose sources, cotton from the cotton plant is almost pure cellulose and is therefore an important source for textiles.

In embodiments, the process for recycling the cotton containing textile includes preparing a solution including at least one enzyme (e.g., a combination of different classes of enzymes such as hemicellulase, glucanase, glucosidase, amylase, and pectinase) and an aqueous solvent. In embodiments, the aqueous solvent maintains the acidity/basicity of the reaction environment at a constant pH. The pH of the solution preferably ranges between 2 to 10 while the molarity of the aqueous solvent preferably ranges between 0.5 to 2. In embodiments, the process further includes depositing the solution into a bioreactor and suspending the cotton containing textile in the solution. An incubation period may range between 5 minutes to 6 months while a bioreactor temperature and a bioreactor pressure may range between 15 to 55 degrees Celsius and 50 to 85 kPa, respectively. In embodiments, parameters such as the temperature within the bioreactor during incubation, the pressure within the bioreactor during incubation, an enzyme concentration, a solvent concentration, an incubation period, and a solution pH may be adjusted depending on the application and to optimize the process. The process may be optimized to achieve a minimum total process time, a maximum end product production, and purity of an end product. For instance, for a 5 cm×5 cm canvas cotton material, an incubation period may be 1 hour at an incubation temperature and pressure of 55 degrees Celsius and 80 kPa, respectively, follow by an incubation period of 20 days at a reduced temperature of 25 degrees Celsius and a reduced pressure of 55 kPa. The solution within which the cotton canvas material is suspended may include IM acetic acid with a mix of protease (50K FCCHUT), papain (50k FCC), bromelain (1800k FCC PU), amylase (12k FCC PU), glucomylase (50 FCC AGU), alpha galactosidase (125 FCC GaIU), lactase (500 FCC ALU), invertase (200 FCC SU), phytase (7 FCC FTU), lyoase (533 FCC LU), cellulase (1.2K FCC CU), beta glucanase (34 FCC BGU), pectase (45 Endo-PG), hemicellulose (3250FCC HCU), and beta xylanse (XU).

In some embodiments, the process further includes washing the cotton containing textile in detergent and/or shredding the cotton containing textile before suspension in the solution. Through biochemical reaction, the at least one enzyme reacts with cellulose in the cotton of the textile during the incubation, biochemically processing the cellulose into other products, of which are water soluble. After incubation, cotton-R, solution, and the other products are output from the bioreactor, wherein cotton-R comprises cotton yarn that is output from the bioreactor after enzymatic bioprocessing of the cotton in the cotton containing textile. In embodiments, the process further includes filtering the cotton-R, the solution, and the other products output from the bioreactor through a filter, separating the other products from the cotton-R and at least some of the solution. In some embodiments, the process further includes returning at least a first portion of the cotton-R and the at least some of the solution filtered through the filter back to the bioreactor for further bioprocessing. In some embodiments, parameters may vary between different incubations (e.g., first incubation, second incubation, etc.), such as the temperature and the pressure within the bioreactor and the incubation period. In some embodiments, the process further includes recovering at least a second portion of the cotton-R after filtration through the filter. In some embodiments, the portion of cotton-R returned to the bioreactor for further bioprocessing and the portion of cotton-R recovered after filtration through the filter is dependent on a level of clumping and a visual inspection of the breakdown of the textile. In some embodiments, the process further includes debundling the recovered cotton-R into cotton fibers, which may then be used a starting material for producing cotton yarn that is usable for textile manufacturing.

Where the textile is a cotton/PET containing textile, the process first includes the cotton/PET containing textile undergoing selective dissolution of PET, whereby the cotton/PET containing textile is suspended in a solvent (e.g., dichloromethane under reflux conditions at a temperature between 20 to 60 degrees Celsius for between 40 minutes to 2 hours or other selective PET soluble solvents at suitable temperatures and treatment times). In some embodiments, the cotton/PET containing textile may be suspended in the solvent at different temperatures and different periods of time than described herein. After selective dissolution of the PET in the solvent, the process further includes extracting cotton leftover after the dissolution of the PET from the cotton/PET containing textile. After extracting the cotton leftover, the process further includes adding alcohol to the solvent within which the PET is dissolved to facilitate precipitation of PET and filtering the combined solvent and alcohol through a filter to extract the precipitated PET (e.g., in a powder form). The recovered PET may then be processed using standard methods to produce PET fibers usable for textile manufacturing. Standard extrusion processes known in the art may then be used to turn the powder into PET fibers for use in, for example, textile manufacturing, building materials, etc. The cotton leftover after the dissolution of PET then undergoes the process for recycling the cotton containing textile as described above to recover cotton-R.

In some embodiments, the at least one enzyme combined with the aqueous solvent to form the solution includes an enzyme that biochemically processes cotton, such as hemicellulase. Different types of enzymes that biochemically process cotton at different rates and/or into different products may be used in different embodiments. In some embodiments, the at least one enzyme is a combination of enzymes. An example of a combination of enzymes that may be used for cellulose-derived textiles include a blend of hemicellulase, glucanase, and glucosidase. In some cases, there may be additional enzymes added, such as amylase or pectinase. These enzymes specifically react with cellulosic material, of which cotton and cotton/PET containing textiles include some form of. For instance, different cellulase enzyme cleaves cellulose at certain bonds to shorten the chain. The biochemical processing that occurs upon suspending the cotton containing textile in the solution includes the breakdown of cellulose into the other products, which are then separated from the cotton-R and the solution returned to the bioreactor and/or the cotton-R recovered via filtration through the filter.

In some embodiments, the process may be operated in a semi-continuous batch mode to extract or recover end products (e.g., cotton-R, the other products) periodically.

In embodiments, the apparatus for recycling the cotton containing textile as described above includes the bioreactor and the filter. The bioreactor is configured for incubation of the cotton containing textile and the solution within which the cotton containing textile is suspended as described above. The filter is configured to at least separate the cotton-R and the solution from the other products output from the bioreactor after the incubation period. In some embodiments, the filter is further configured to separate the portion of the cotton-R that are to be recovered from the portion of the cotton-R that are to be returned to the bioreactor for further bioprocessing. In some embodiments, the apparatus further includes a means for transferring the outputs of the bioreactor to the filter for filtration. In some embodiments, the apparatus further includes a means for returning at least a portion of the cotton-R filtered through the filter to the bioreactor for further bioprocessing. In some embodiments, the apparatus is a small scale unit and/or a mobile unit.

Where the textile contains cotton/PET, the apparatus further includes the vessel for containing the solvent for selective dissolution of PET, the filter for filtering the combination of the solvent within which the PET is dissolved and alcohol to extract precipitated PET as described above, and a means for transferring the combined solvent within which the PET is dissolved and alcohol to the filter for filtration.

In different embodiments, at least one step of the process for recycling cotton and cotton/PET containing textiles may be rearranged, removed, altered, and/or added.

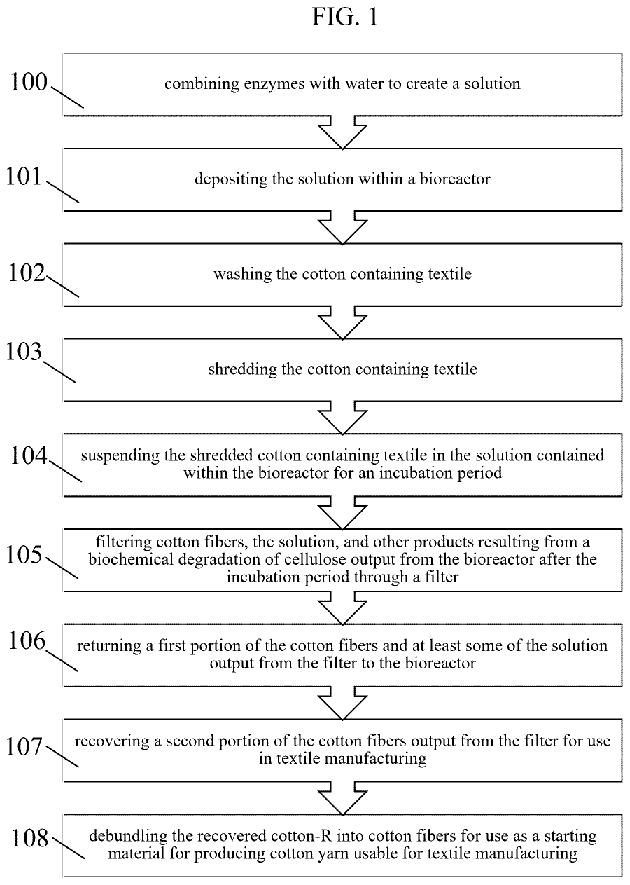

FIG. 1 illustrates an example of a process for recycling a cotton containing textile including: combining enzymes with water to create a solution 100; depositing the solution within a bioreactor 101; washing the cotton containing textile 102; shredding the cotton containing textile 103; suspending the shredded cotton containing textile in the solution contained within the bioreactor for an incubation period 104; filtering cotton-R, the solution, and other products resulting from biochemical processing of cellulose output from the bioreactor after the incubation period through a filter 105; returning a first portion of the cotton-R and at least some of the solution output from the filter to the bioreactor 106; and recovering a second portion of the cotton-R output from the filter 107; and debundling the recovered cotton-R into cotton fibers for use as a starting material for producing cotton yarn usable for textile manufacturing 108. Upon reacting with the enzymes in the solution during the incubation period, the cotton containing textile undergoes a biochemical process, wherein cellulose of the cotton in the textile is broken down into the other products, of which are water soluble and separated from the cotton-R via filtration through the filter.

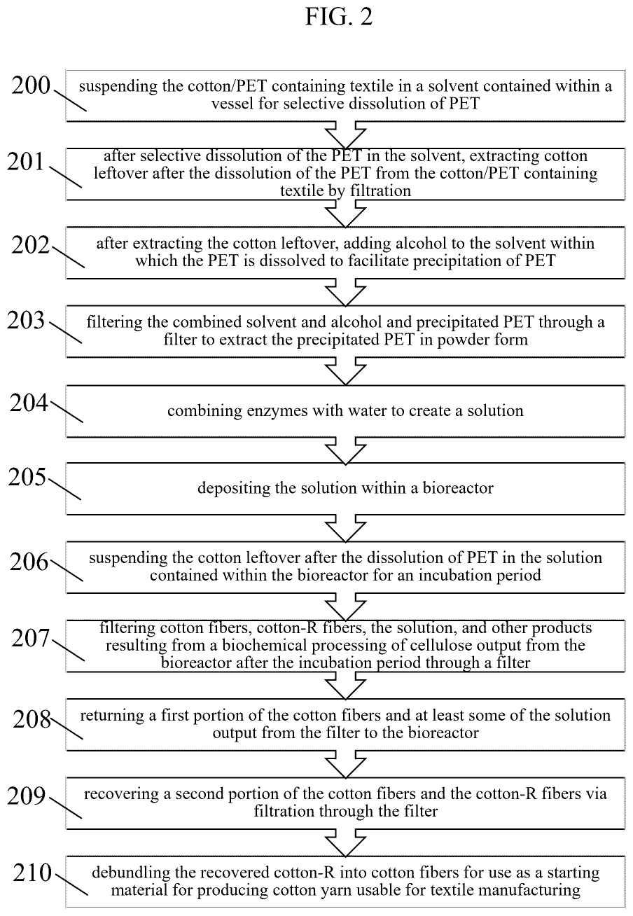

FIG. 2 illustrates an example of a process for recycling a cotton/PET containing textile including: suspending the cotton/PET containing textile in a solvent contained within a vessel for selective dissolution of PET 200; after selective dissolution of the PET in the solvent, extracting cotton leftover after the dissolution of the PET from the cotton/PET containing textile by filtration 201; after extracting the cotton leftover, adding alcohol to the solvent within which the PET is dissolved to facilitate precipitation of PET 202; filtering the combined solvent and alcohol and precipitated PET through a filter to extract the precipitated PET 203; preparing a solution comprised of water and enzymes 204; depositing the solution within a bioreactor 205; suspending the cotton leftover after the dissolution of PET in the solution contained within the bioreactor for an incubation period 206; filtering cotton-R, the solution, and other products resulting from a biochemical processing of cellulose output from the bioreactor after the incubation period through a filter 207; returning a first portion of the cotton-R and at least some of the solution output from the filter to the bioreactor 208; recovering a second portion of the cotton-R via filtration through the filter 209; and debundling the recovered cotton-R into cotton fibers for use as a starting material for producing cotton yarn usable for textile manufacturing 210.

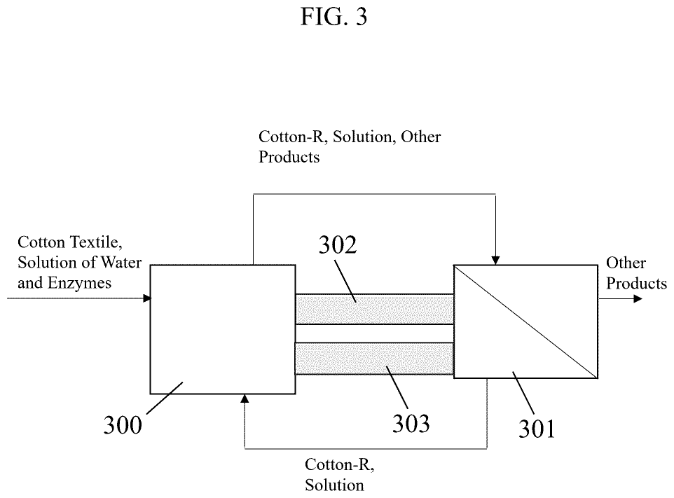

FIG. 3 illustrates an example of an apparatus for recycling the cotton containing textile as described in the process of FIG. 1. The apparatus includes a bioreactor 300 for incubation of the cotton containing textile and a solution within which the cotton containing textile is suspended; a filter 301 for filtering cotton-R, the solution, and other products output from the bioreactor after the incubation; a first means 302 for transferring the cotton-R, the solution, and the other products from the bioreactor after the incubation to the filter 301 for filtration; and a second means 303 for returning the cotton-R and at least the portion of the solution filtered through the filter 301 back to the bioreactor 300.

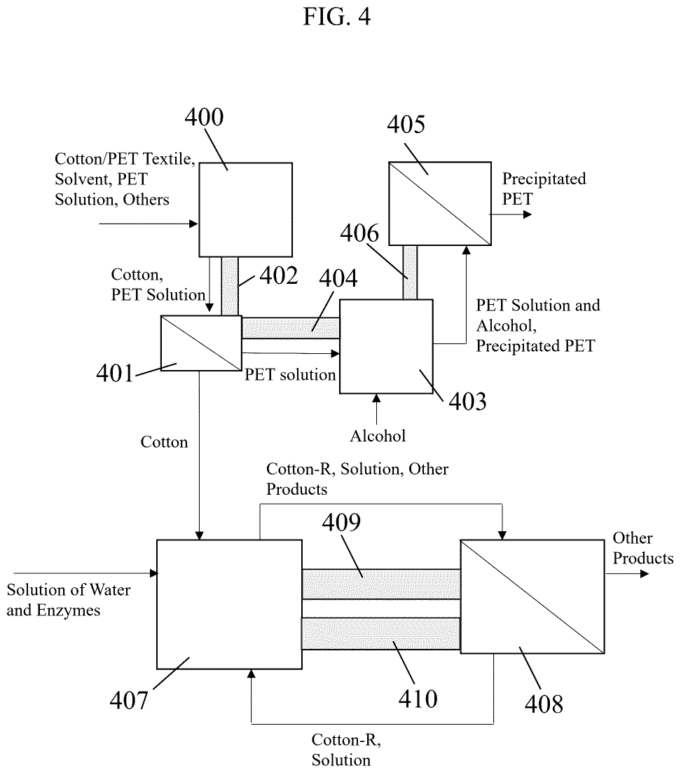

FIG. 4 illustrates an example of an apparatus for recycling the cotton/PET containing textile as described in the process of FIG. 2. The apparatus includes a vessel 400 for containing the cotton/PET containing textile, a solvent (e.g., dichloromethane) for selective dissolution of PET (leaving cotton of the cotton/PET containing textile), PET solution, and others (e.g., additives); a first filter 401 for separating the cotton from the PET solution by filtration; a first means 402 for transferring cotton (in solid form) left over after the dissolution of PET and the PET solution from the first vessel 400 to the first filter 401; a second vessel 403 within which a non-solvent (e.g., isopropyl alcohol) is combined with the PET solution output from the first filter 401 to precipitate PET (e.g., in granular form); a second means 404 for transferring the PET solution from the first filter 401 to the second vessel 403; a second filter 405 for separating the precipitated PET from the combined non-solvent and PET solution by filtration; a third means 406 for transferring the combined non-solvent and PET solution and precipitated PET to the second filter 405 for filtration; a bioreactor 407 for incubation of cotton that is leftover after the dissolution of PET and a solution including enzymes within which the cotton that is leftover after the dissolution of PET is suspended; a third filter 408 for filtering cotton-R, the solution, and other products output from the bioreactor 407 after the incubation (cotton-R is solid and therefore does not pass through the third filter 408); a fourth means 409 for transferring the cotton-R, the solution, and the other products from the bioreactor 407 after the incubation to the second filter 408 for filtration; and a fifth means 410 for returning the cotton-R and at least the portion of the solution filtered through the second filter 408 back to the bioreactor 407. Cotton-R produced may circulate periodically from the bioreactor 407 to the second filter 408 for recovery. In some embodiments, the cotton is washed and cleaned prior to suspension in the enzymatic solution contained within the bioreactor.

The apparatus and process for recycling cotton and cotton/PET containing textile described herein is more environmentally friendly than some chemical recycling processes known in the art, as enzymes are used rather than harsh chemicals. Further, the process for recycling cotton and cotton/PET containing textiles recovers high-quality cotton and PET fibers that can be used to create new textile products, reducing the need for virgin cotton and PET material.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope.

1. A process for recycling a textile, comprising:
combining at least one enzyme with an aqueous solvent, their combination resulting in a creation of a solution;
depositing the solution within a bioreactor;
suspending the textile in the solution within the bioreactor for an incubation period, wherein:
the textile comprises at least cotton;
during the incubation period the at least one enzyme biochemically reacts with cellulose of the cotton in the textile, thereby breaking down the cellulose and producing other products; and
cotton yarn, the solution, and the other products are output from the bioreactor after the incubation period;
filtering the cotton yarn, the solution, and the other products output from the bioreactor after the incubation period through a first filter, separating at least the other products from the cotton yarn and at least some of the solution;
recovering at least a first portion of the cotton yarn filtered through the first filter; and
returning any portion of the cotton yarn and the at least some of the solution filtered through the first filter back to the bioreactor when further processing of the any portion of the cotton yarn is required.

2. The process of claim 1, further comprising:
debundling the recovered cotton yarn into cotton fibers.

3. The process of claim 1, further comprising:
washing the textile with a detergent prior to suspending the textile in the solution.

4. The process of claim 1, further comprising:
shredding the textile prior to suspending the textile in the solution.

5. The process of claim 1, wherein:
the cotton yarn is water insoluble; and
the other products are water soluble.

6. The process of claim 1, wherein:
an initial textile initially comprises polyester (PET) and the cotton;
the process first comprises:
suspending the initial textile in a solvent contained within a vessel for selective dissolution of the PET;
extracting cotton leftover after the dissolution of the PET from the initial textile;
after extracting the cotton leftover, adding alcohol to the solvent within which the PET is dissolved to facilitate precipitation of PET; and
filtering the combined solvent and alcohol through a second filter to extract the precipitated PET; and
the textile comprises the cotton leftover after the dissolution of the PET from the initial textile.

7. The process of claim 6, wherein the initial textile is suspended in the solvent for selective dissolution of PET under reflux conditions.

8. The process of claim 6, wherein the initial textile is suspended in the solvent for selective dissolution of PET:
at a temperature between 20 to 60 degrees Celsius; and
for between 40 minutes to 2 hours.

9. The process of claim 1, wherein the process is operated in a semi-continuous batch mode to continuously recover at least cotton yarn periodically.

10. The process of claim 1, wherein the at least one enzyme comprises at least one of: a blend of cellulase, hemicellulase, glucanase, glucosidase, amylase, and pectinase.

11. The process of claim 1, wherein:
the incubation period is between 5 minutes to 6 months;
a temperature within the bioreactor during the incubation period is between 15-55 degrees Celsius; and
a pressure within the bioreactor during the incubation period is between 50-85 kPa.

12. The process of claim 1, wherein:
a pH of the solution is between 2 to 10; and
a molarity of the aqueous solvent is between 0.5-2.

13. An apparatus for recycling a textile, comprising:
a bioreactor configured for incubation of the textile and a solution comprising at least one enzyme and an aqueous solvent within which the textile is suspended for an incubation period at a temperature and a pressure, wherein:
the textile comprises at least cotton;
during the incubation period the at least one enzyme biochemically reacts with cellulose of the cotton in the textile, thereby breaking down the cellulose and producing other products; and
cotton yarn, the solution, and the other products are output from the bioreactor after the incubation period;
a first filter configured to filter the cotton yarn, the solution, and the other products output from the bioreactor after the incubation period, separating at least the other products from the cotton yarn and at least some of the solution;
wherein:
at least a first portion of the cotton yarn filtered through the first filter are recovered; and
any portion of the cotton yarn and the at least some of the solution filtered through the first filter are returned to the bioreactor when further processing of the any portion of the cotton yarn is required.

14. The apparatus of claim 13, wherein the recovered cotton yarn is debundled into cotton fibers.

15. The apparatus of claim 13, wherein the textile is washed with a detergent prior to suspending the textile in the solution.

16. The apparatus of claim 13, wherein the textile is shredded prior to suspending the textile in the solution.

17. The apparatus of claim 13, wherein:
an initial textile initially comprises PET and cotton;
the apparatus further comprises:
a vessel for containing a solvent within which the initial textile is suspended for selective dissolution of the PET, wherein:
cotton leftover after the dissolution of the PET from the initial textile is extracted; and
after extracting the cotton leftover, alcohol is added to the solvent within which the PET is dissolved to facilitate precipitation of PET; and
a second filter for filtering the combined solvent and alcohol to extract the precipitated PET; and
the textile comprises the cotton leftover after the dissolution of the PET from the initial textile.

18. The apparatus of claim 17, wherein the initial textile is suspended in the solvent for selective dissolution of PET:
under reflux conditions;
at a temperature between 20 to 60 degrees Celsius; and
for between 40 minutes to 2 hours.

19. The apparatus of claim 13, wherein the at least one enzyme comprises at least two of:
a blend of cellulase, hemicellulase, glucanase, glucosidase, amylase, and pectinase.

20. The apparatus of claim 13, wherein:
the incubation period is between 5 minutes to 6 months;
a temperature within the bioreactor during the incubation period is between 15-55 degrees Celsius;
a pressure within the bioreactor during the incubation period is between 50-85 kPa;
a pH of the solution is between 2 to 4; and
a molarity of the solvent is between 0.5-2.
